(19) United States
(12) Patent Application Publication
(10) Pub. No.: US 2021/0401395 A1
(43) Pub. Date: Dec. 30, 2021

(54) Audible Handheld Stethoscope

(71) Applicants: Rabiatu Kamara, Lorton, VA (US); Abdul Bangura, Lorton, VA (US)

(72) Inventors: Rabiatu Kamara, Lorton, VA (US); Abdul Bangura, Lorton, VA (US)

(21) Appl. No.: 17/134,754

(22) Filed: Dec. 28, 2020

Related U.S. Application Data
(60) Provisional application No. 63/045,324, filed on Jun. 29, 2020.

(51) Int. Cl. A61B 7/04; A61B 5/00

(52) U.S. Cl. CPC A61B 7/04; A61B 5/7225; A61B 2560/0214; A61B 2560/0406; A61B 2560/0431; A61B 5/0004

(57) ABSTRACT

This present invention relates to a tubeless audible handheld stethoscope without integrated earpieces. The stethoscope has a receiving portion and a processing portion. The processing portion includes a built-in speaker and a clip assembly. The built-in speaker emits the sound of the stethoscope to a medical professional when placed over an area of the part of the patient. The stethoscope allows the medical professional to monitor a patient at a distance and without risking exposure to infection, as well as eliminating the cumbersome wearing of traditional stethoscopes.

### CROSS-REFERENCE TO RELATED APPLICATION

The present application claims priority to, and the benefit of, U.S. Provisional Application No. 63/045,324, which was filed on Jun. 29, 2020 and is incorporated herein by reference in its entirety.

### FIELD OF THE INVENTION

The present invention relates generally to the field of diagnostic instruments. More specifically, the present invention relates to a medical device capable of issuing an audible sound which allows the medical professionals to listen to the vital signs of a patient without the use of traditional earpieces provided with stethoscopes. The audible handheld stethoscope is comprised of a speaker that emits the vital sound information of body organs of a patient, such as the heart, upon which the audible handheld stethoscope is placed. The audible handheld stethoscope further comprises a clip assembly to attach the stethoscope onto a medical gown, scrubs, a medical coat or the like. Accordingly, the present disclosure makes specific reference thereto. However, it is to be appreciated that aspects of the present invention are also equally usable with other like applications, devices and methods of manufacture.

### BACKGROUND OF THE INVENTION

By way of background, the concept of using a stethoscope by medical personnel to listen to the vitals and other body's internal sound (auscultation) is well known. Auscultation techniques are useful for medical examination, testing, and diagnosis purposes and auscultation is normally performed to examine select biological systems, such as the cardiovascular, respiratory, and/or gastrointestinal systems of a patient or animal. A conventional acoustic stethoscope conveys sounds from inside a patient to the ears of a physician, health-care provider or other medical professional via acoustic tubes that isolate the signal. The acoustic tubes are coupled to the patient's body through a bell that may optionally be covered by an acoustic diaphragm. This allows the physician, health-care provider or other medical professional to quickly identify whether the patient is experiencing health issues.

The conventional stethoscope is generally worn around the neck of a physician or health care professional for prolonged periods of time, which may result in neck and back pain. Conventional stethoscopes can also be heavy, bulky, and inconvenient to transport, and may extend outwardly from the medical professional's pocket, thereby potentially getting caught on surrounding items.

Additionally, conventional stethoscopes may also be a source of the transfer of infectious diseases. More specifically, conventional stethoscopes are used on nearly all patients to monitor cardiac, pulmonary or digestive sounds, but they can also expose physicians, other health care providers and patients to infectious diseases via the earpieces and sound tubes. Further, due to limited length of the sound tubes of a conventional stethoscope, a physician has to sit very near to a patient to use the stethoscope, thereby exposing himself or herself to infectious diseases or pathogens from an infected patient. Likewise, the patient may be exposed if the health care professional is unknowingly contagious at the time of examination.

Many times a stethoscope is also shared among various physicians or other health care providers, thereby causing bacteria and other pathogens to move from one physician to the next. Studies have shown that a relationship exists between the usage of stethoscopes and the occurrence of external otitis and colonization of the stethoscope's earpiece with microorganisms that possess the potential for causing nosocomial infection. While increased washing and/or sterilization of the stethoscope tubes and earpieces may reduce nosocomial infections, this is often not practical in fast-paced or emergency medical environments. Further, cleaning practices for assessment tools, such as stethoscopes, are erratic and may depend on the user, health care provider or others. Also, the sound tubes used to transmit acoustic sounds through traditional stethoscopes can create extraneous noise when the tubes rub against the hands, body, or clothing, etc., thereby causing distortion or difficulty in hearing and identifying the faint sounds that can be made by the body organs, particularly if the patient is suffering from a disease or an injury.

Therefore, there exists a long felt need in the art for a handheld stethoscope that can be used by a physician, healthcare provider or other medical professional to auscultate a patient without the need to use earpieces and sound tubes, which may be unreliable. There is also a long felt need for a handheld stethoscope that emits sounds from a patient's vitals and internal organs in an amplified manner using a speaker, such that the vitals can be heard clearly at a distance from the patient that is further than when a traditional stethoscope is used. Moreover, there is a long felt need in the art for a handheld stethoscope device that reduces the likelihood of patient to physician or physician to patient infectious disease transmission that is associated with the use of traditional stethoscopes. Additionally, there is a long felt need in the art for a handheld stethoscope that is compact in size, lightweight and can be carried easily like a pen in a pocket, without leading to neck and/or back pain that can be caused by the wearing of a traditional stethoscope around the neck for prolonged period of times. Moreover, there is a long felt need in the art for a handheld stethoscope that does not require the use of auxiliary earpieces or connecting tubes, and that has wireless capabilities, thereby enabling the device to be connected to a smartphone application to graphically display the vitals, or to listen to the sounds remotely. Finally, there is a long felt need in the art for a portable handheld stethoscope that is relatively inexpensive to manufacture, and both safe and easy to use.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, discloses a portable handheld stethoscope that has an internal speaker to play and emit the vitals and internal organ sounds of a patient to allow a physician, healthcare worker or other medical professional to perform auscultation without the need for sound tubes and/or earpieces. The handheld audible stethoscope further comprises a microphone that detects vitals or organ sounds, an amplifier to amplify the detected sound to a desired level, a filter to remove the noise from the detected sound such that the noise is out of the desired wavelength and a transmission circuit to transmit the filtered vitals and body sounds to the speaker to play the sound with sufficient audible clarity.

In a further embodiment, the portable handheld audible stethoscope may also comprise a clip assembly to allow a medical professional to clip the device to their scrubs, clothing, medical coat, or other garment, as well as a wireless communication module, thereby enabling a wireless communication technology such as Bluetooth, Wi-Fi, Infrared or Wi-Fi Direct, etc. to transfer the vitals and other body organ sounds to a connected smartphone, wireless earphone, PDA, laptop, or the like. The stethoscope has a battery or power source for powering the device, and can be turned on/off using a control on the stethoscope.

The subject matter disclosed and claimed herein, in another embodiment thereof, comprises an audible handheld stethoscope used by medical professionals for auscultation. The audible handheld stethoscope is comprised of a stethoscope head, a processing portion having a proximal end and a distal end, a speaker positioned at the distal end of the processing portion, and a connecting tube connecting the stethoscope head and the proximal end of the processing portion. The stethoscope head has a diaphragm and a bell to pick up vibrating sensations from the patient's body, a microphone to hear vitals and other organ sounds from the vibrating sensations, and passing the sound signals from the stethoscope head to the processing portion through the connecting tube. The processing portion is further comprised of an amplifier to amplify the received sound signal to a desired level and wavelength range, a filter for removing unwanted noise from the detected sound, and a transmission circuit for transmitting the filtered vitals and body sounds to the speaker to be played aloud. The audible handheld stethoscope may further comprise a clip or other supporting assembly on the top surface of the processing portion to allow the stethoscope to be clipped to a pocket, a medical coat or other garment.

In another embodiment of the present invention, a tubeless handheld stethoscope is disclosed and is comprised of an integrated speaker at a distal end of the tubeless handheld stethoscope to emit the vitals and other body sounds detected by the stethoscope head. More specifically, the speaker outputs a human audible sound that allows the medical professional to hear and interpret the vitals clearly and distinctly. More importantly, the sound tubes and earpieces used in conventional stethoscopes, and all of their inherent limitations, are not required in order to hear the vitals and other body sounds in the tubeless handheld stethoscope of the present invention.

In yet another embodiment of the present invention, a tubeless handheld stethoscope is disclosed to perform auscultation. The tubeless handheld stethoscope has a contact microphone that picks up vibrations directly from the body organ over which the stethoscope head is placed. The contact microphone includes a magnetic transducer, a contact pin and a contact plate. The microphone detects vitals and other body sounds, which are amplified and filtered and sent to an integrated speaker to be played in human audible sound.

In a further embodiment of the present invention, a tubeless auscultation device is disclosed and includes a processing device that has an amplifier to amplify a body organ sound signal detected by a microphone present in the stethoscope head to a desired level and wavelength range. An associated filter removes the noise from the detected sound such that if the noise signal does not fall within the desired wavelength range it is filtered out and a transmission circuit transmits the filtered vitals and body sound to an integrated speaker to play the detected sound aloud. The processing device has a wireless communication module enabling a wireless communication technology, such as Bluetooth, Wi-Fi, Infrared, Wi-Fi Direct to transfer the vitals and other body organ sounds from the tubeless auscultation device to a connected smartphone, wireless earphone, a PDA or the like.

In yet another embodiment of the present invention, a method of performing auscultation using a tubeless handheld stethoscope of the present invention is disclosed and comprises the initial step of turning on the stethoscope by using a control button present on the stethoscope body, and then placing the stethoscope head against a person's body and positioned over a source of auscultation, such as the patient's heart or lungs. Next, the captured auscultation signal is converted into a sound signal, and the sound signal is amplified to a desired power level. Undesired noise signals are then filtered out from the sound signals, and are transmitted to a built-in speaker to play the vitals and other body organ sounds as a human audible sound. The stethoscope is further comprised of a memory to store the captured auscultation signals to be later analyzed by a physician or other medical professional.

In a still further embodiment of the presently described invention, a tubeless stethoscope for performing auscultation is described and includes a built-in speaker to play the auscultation sound in real time, or to playback the auscultation from a memory in a human audible sound. The stethoscope is relatively lightweight and compact, and does not have bulky sound tubes and earpieces typically associated with prior art stethoscopes. The stethoscope may connect to a remote speaker, such as wireless headphones, smartphones, PDAs, laptop and the like, through a wireless technology to transmit the auscultation signal to the remote speaker for the medical professional to hear. Once the auscultation is performed, the stethoscope may be turned off and can be clipped on to a pocket of a shirt or coat until further needed.

In yet a further embodiment of the presently described invention, a tubeless stethoscope is disclosed and comprises a replaceable stethoscope head that is removably secured with a connecting tube. To avoid infection among various patients, the head can be disposed of once it has been used for a patient, and can be replaced easily with a new head by a physician, nurse or other individual. The auscultation sound from the head is output through a built-in speaker.

In its various embodiments, the tubeless audible handheld stethoscope of the present invention is ergonomically designed such that it can be utilized with a single hand, and is comprised of a nonmetallic contact surface to be placed in contact with a patient. Additionally, the stethoscope head attachment used in the present invention is engineered to minimize noise causing vibrations and includes a microphone that is positioned to optimize sensitivity for listening to and recording internal body sounds. The various components of the stethoscope of the present invention are constructed of materials that are easy-to-clean, waterproof or water resistant, and are not cold to the touch.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a top perspective view of one potential embodiment of the audible handheld stethoscope of the present invention in accordance with the disclosed architecture;

FIG. 2 shows a schematic view of the various components present in the processing portion of one potential embodiment of the audible handheld stethoscope of the present invention for processing the vibrations and sounds emanating from a body organ in accordance with the disclosed architecture;

FIG. 3 illustrates a top perspective view of one potential embodiment of the audible handheld stethoscope of the present invention in accordance with the disclosed architecture wherein the built-in speaker and the attached clip assembly are clearly visible;

FIG. 4 illustrates a bottom perspective view of one potential embodiment of the audible handheld stethoscope of the present invention in accordance with the disclosed architecture;

FIG. 5 illustrates a perspective view of a medical professional using one potential embodiment of the audible handheld stethoscope of the present invention to analyze a patient in accordance with the disclosed architecture; and

FIG. 6 illustrates a perspective view of one potential embodiment of the audible handheld stethoscope of the present invention clipped to the pocket of a garment being worn by a medical professional.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an audible handheld stethoscope that can be used by a physician to auscultate a patient without the need to use earpieces and sound tubes, and that emits sounds from a patient's vitals and internal organs in an amplified manner via a speaker. Moreover, there is a long felt need in the art for an audible handheld stethoscope device that reduces the likelihood of patient to physician (or vice versa) infectious disease transmission, is relatively compact in size, lightweight and can be carried easily in a medical professional's pocket. Further, there is a long felt need in the art for an audible handheld stethoscope that does not require the use of auxiliary earpieces or connecting tubes, and that has wireless capabilities, thereby enabling the device to be connected to a smartphone application to graphically display the vitals, or to listen to the sounds remotely.

The present invention, in one exemplary embodiment, is an audible handheld stethoscope used by medical professionals for auscultation. The audible handheld stethoscope is comprised of a stethoscope head, a processing portion having a distal end and a proximal end, a speaker located at the distal end of the processing portion, and a connecting tube connecting the stethoscope head and the proximal end of the processing portion. The stethoscope head is comprised of a diaphragm and a bell to detect vibrating sensations emanating from a body organ, a microphone to detect vitals and other organ sounds from the vibrating sensations, which are passed from the stethoscope head to the processing portion through the connecting tube. The processing portion is further comprised of an amplifier to amplify the received sound signals to a desired level and wavelength range, and a filter to remove the background and other noises from the detected sound, such that if the noise signal does not fall within the desired wavelength range, it is filtered out. A transmission circuit is used to transmit the filtered vitals and body sounds to the integrated speaker to be played aloud for the medical professional. The audible handheld stethoscope may further comprise a memory for recording the sounds, and a clip assembly on the top surface of the processing portion to allow the stethoscope to be clipped to a pocket, belt or garment.

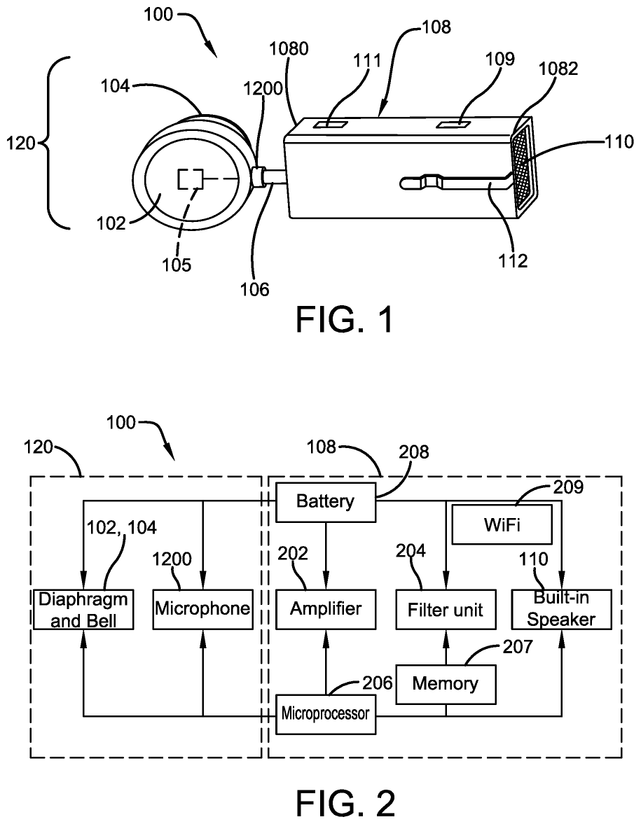

Referring initially to the drawings, FIG. 1 illustrates a top perspective view of one potential embodiment of the audible handheld stethoscope 100 of the present invention in accordance with the disclosed architecture. More specifically, the audible handheld stethoscope 100 is comprised of a stethoscope head 120, a processing portion 108, a connecting tube 106, and a built-in speaker 110. The stethoscope head 120 is comprised of a diaphragm 102 and a bell 104 that are placed in contact with a patient and over the body organ area to be diagnosed, and a microphone 105, such as a MEMS microphone or contact microphone, to pick up sound signals from the body of the patient and/or to convert vibration signals to auscultation sound signals. More specifically, the stethoscope head 120, through diaphragm 102 and/or bell 104, detects vibrations in the patient's body representing auscultation signals. The diaphragm 102 is a sealed membrane that vibrates, and detects higher pitched sounds, like breath sounds and normal heart beat sounds. In turn, the bell 104 detects lower pitched sounds, like some heart murmurs and some bowel sounds. The signals from the stethoscope head 120 are then transmitted to the processing portion 108 through the connecting tube 106 for further processing of the auscultation sound signals.

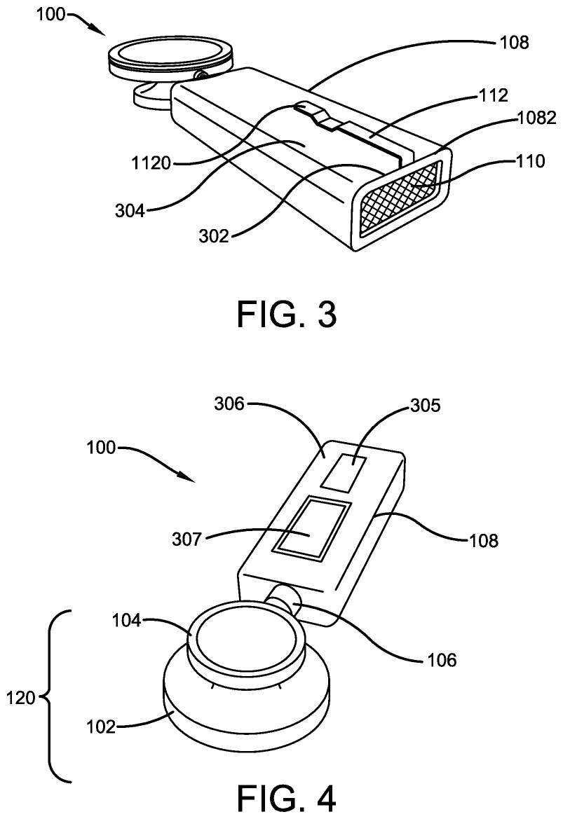

The processing portion 108 is comprised of a proximal end 1080 and a distal end 1082. The proximal end 1080 of the processing portion 108 is the portion closest to the stethoscope head 120, and connected thereto by the connecting tube 106. The distal end 1082 of the processing portion 108 is the portion furthest away from the stethoscope head 120, and the built-in speaker 110 is positioned on the distal end 1082. The processing portion 108 employs electronic technology and circuitry with signal processing capabilities that enable amplification, filtering of detected sounds, transmission to a speaker, etc. The processing portion 108 receives the signals detected by the diaphragm 102 and/or the bell 104 of the stethoscope head 120 via connecting tube 106 and processes the same. After processing the signals, the processing portion 108 causes the built-in speaker 110 present at the distal end 1082 of the processing portion 108 to emit the sound in human audible form. The sound emitted from the speaker 110 is sharp, crisp, and clear, thereby enabling better diagnosis of the patient's condition. The speaker 110 produces signals enhanced to approximately 60 to 100 times that of traditional stethoscopes. These values of enhancement, however, are presented for illustration only, and not by way of limitation. As best shown in FIGS. 1 and 3, the processing portion 108 may also comprise a clip or other fastening assembly 112 to clip the stethoscope 100 to a medical professional's scrubs, pocket, belt, clothing or the like.

In a preferred embodiment, the audible handheld stethoscope 100 may further comprise a power source, such as a disposable or rechargeable battery 208 to power the stethoscope 100 and the various electronic components present in the processing portion 108. The batteries 208 can preferably provide uninterrupted operation of the audible handheld stethoscope 100 for up to 20 hours, and the stethoscope 100 may be turned off automatically if not in use for a predetermined period of time (e.g., two to five minutes) to conserve battery power. Additionally, eliminating the sound tubes and earpieces used in traditional stethoscopes allows the physician or other medical professional to be free of the tubes and the close proximity to the patient, while still remaining in the room and attending to the patient during the procedure. Further, other medical professionals can hear the auscultation sounds from a further distance to assist with assessing the patient's condition.

FIG. 2 shows a schematic view of the various components present in the processing portion 108 of one potential embodiment of the audible handheld stethoscope 100 of the present invention for processing the vibrations and sounds emanating from a body organ in accordance with the disclosed architecture. As noted above, the stethoscope head 120 has a diaphragm 102 and a bell 104, which is placed in proximity to the body organ of the patient to be analyzed. The diaphragm 102 detects higher pitched sounds, like those emanating from the patient's lungs and heart, and the bell 104 detects lower pitched sounds, like some associated with a heart murmur, bowel sounds, and the like. The vibrations are also detected by a microphone 1200 present within the stethoscope head 120, which converts the vibrations into sound signals. The sound signals are then transmitted to the processing portion 108 through a connecting tube 106. In the processing portion 108, the sound signals are amplified to a power level (i.e., a specific wavelength range of a radio frequency signal) by an amplifier 202. The amplified signals are then transmitted to a filter unit 204 to remove unwanted noise signals, such as background noise, bodily noise not present in the specific wavelength range, etc. to smooth and clarify the amplified signals. The filtered signals are then emitted through the built-in speaker 110 present at the distal portion 1082 of the processing portion 108.

As also noted above, the processing portion 108 may also have a battery 208 to provide energy and power to the various components and circuitry present in the processing portion 108 and the stethoscope head 120. The battery 208 may be a conventional nickel cadmium battery, rechargeable battery or other suitable energy supply source. If the audible handheld stethoscope 100 is provided with a rechargeable battery, a port 109 may also be provided so that the battery 208 can be recharged as needed. The processing portion 108 also comprises a microprocessor 206 to digitize the signals (if required), and control the timing signals and operations of the components and circuitry of the audible handheld stethoscope 100. The microprocessor 206 may further comprise a power monitoring function to turn off the device after a period of inactivity to conserve battery life. The microprocessor 206 may alternatively be coupled to a timer unit (not shown) which provides the same shut down activity. The sound emitted from the speaker 110 is in a human audible range, such that the sound is clear, distinct and crisp to allow a medical professional to correctly detect the condition of the patient. The processing portion 108 may also include a USB port 111 so that a flash drive or other connection with a laptop or smart device can be made for downloading information from the memory 207, as best shown in FIG. 2.

In should be appreciated that the stethoscope 100 may have additional components to enhance the functionality and efficiency of auscultation. In one embodiment, a memory 207 may be present in the processing portion 108 to store the auscultation data. In another embodiment, the processing portion 108 may have a Wi-Fi or Bluetooth module 209 to wirelessly transmit the auscultation data to a wireless device such as a smartphone, PDA, laptop or the like. This embodiment is particularly beneficial as it enables a physician or other medical professional who is not physically present at the location where the auscultation is performed to hear the sounds emitted from the built-in speaker 110. An LED may also be present to indicate the status of the operation of the stethoscope 100. As an example, the LED may turn “GREEN” when the auscultation is being performed, and a “RED” LED indication may indicate a low battery level.

FIG. 3 illustrates the built-in speaker 110 and the attached clip assembly 112 of the audible handheld stethoscope 100 of the present invention in accordance with the disclosed structure. More specifically, the speaker 110 is present at the distal end 1082 of the processing portion 108, and may include commercially available devices, such as a separate tweeter and woofer to augment and/or play an audible sound. The speaker 110 volume can be turned on, off, up or down as per the control through the microprocessor present in the processing portion 108.

The spring clip assembly 112 is hingedly attached at the middle 302 of the distal end 1082 of the processing portion 108, and is used to clip the audible stethoscope 100 to the medical provider's pocket, belt, clothing, medical coat, etc. A physician need not to carry the stethoscope 100 of present invention around his or her neck, since sound tubes and earpieces are not needed with the audible handheld stethoscope 100 of the present invention. In use, when the free end 1120 of the clip assembly 112 is released, the clip assembly springs back such that the clothing (pocket sleeve) is held between the clip 112 and the top surface 304 of the processing portion 108. In an alternative embodiment, the clip assembly 112 may be replaced with a simple hook which is molded to the top surface 304 of the processing portion 108 at the middle point 302 of the distal end 1082.

FIG. 4 illustrates a bottom perspective view of one potential embodiment of the audible handheld stethoscope 100 of the present invention in accordance with the disclosed architecture. More specifically, the bottom surface 306 of the processing portion 108 is preferably a relatively smooth surface, and on the same side of the bell 104 on the stethoscope head 120. The bottom surface 306 may further comprise a lid 305 for accessing the rechargeable battery 208 in the processing portion 108, as well as an LED display 307 that can display images corresponding to the vibration signals, as well as the status of the audible handheld stethoscope 100.

As noted, the audible handheld stethoscope 100 is relatively compact, lightweight, and without the sound tubes and earpieces associated with prior art stethoscope device. The audible handheld stethoscope 100 is also comprised of a durable, moisture resistant and non-porous casing so as to allow the stethoscope 100 to be easily carried by a practitioner without causing damage to the internal components. The individual elements of the stethoscope 100 may be formed together as one continuous element, either through manufacturing processes, such as welding, casting, or molding, or through the use of a singular piece of material milled or machined with the aforementioned components forming identifiable sections thereof.

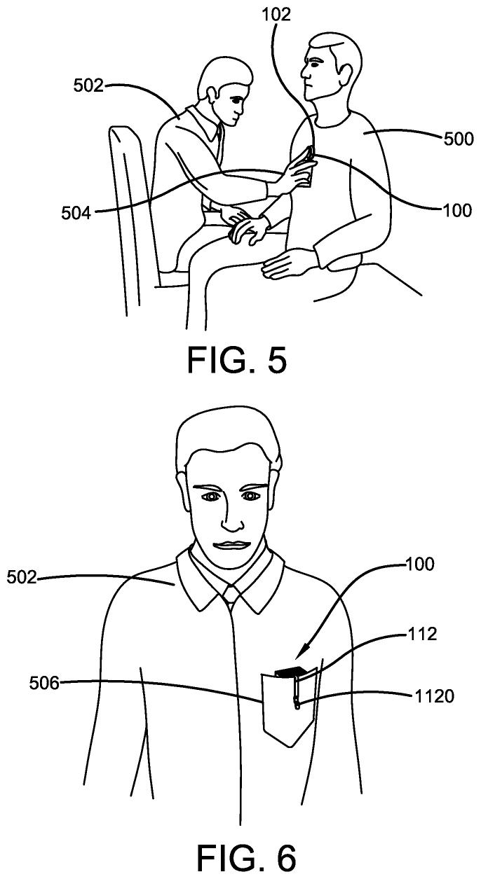

FIG. 5 illustrates a perspective view of a medical professional 502 using one potential embodiment of the audible handheld stethoscope 100 of the present invention to analyze a patient 500 in accordance with the disclosed architecture. More specifically, a medical professional 502 uses the audible stethoscope 100 by placing the stethoscope head 120 and the diaphragm 102 over the portion of the patient's body 500 to be analyzed. The audible stethoscope 100 detects the sound signal produced by the patient's body 500, amplifies and processes it via the processing portion 108, and then broadcasts the same through the built-in speaker 110. In this manner, the correct sound signal is emitted by the speaker 110, thereby allowing the medical professional 502 to make the correct diagnosis after listening to the auscultation sound.

As clearly shown, the audible stethoscope 100 is relatively compact and can be operated using a single hand 504 of the medical professional 502. Further, no earpiece is worn by the physician 502 and no bulky sound tubes are integrated with the stethoscope 100. Simultaneous with the broadcasting of the vitals, the stethoscope 100 records physiological data in the integrated memory 207 (FIG. 2), and may also transmit the data to a physician's or other personnel's computer or portable electronic device.

FIG. 6 illustrates a perspective view of one potential embodiment of the audible handheld stethoscope 100 of the present invention clipped to the pocket 506 of a garment being worn by a medical professional 502. More specifically, the audible stethoscope 100 of the present invention is clipped onto the pocket 506 using the clip assembly 112, wherein the free end 1120 of the clip assembly 112 is applied to the clothing (i.e., pocket sleeve). The clip assembly 112 may also be attached to a waistband, tote bag, belt, purse, or the like.

In one embodiment, the audible stethoscope 100 of the present invention may have an analog and/or digital display to show a graphical image of the electrical auscultation signal to the physician. The display may include a screen incorporated into the stethoscope 100, and the data may be shown numerically or graphically, depending on the display mode selected by the user.

In another embodiment, the audible stethoscope 100 may be available with a dedicated software application, such as a mobile application that allows the medical professional to, for example, hear the real time bodily sounds transmitted from a patient, delayed bodily sounds transmitted from a patient or previously recorded bodily sounds transmitted from a patient. The application may have the functionality that allows the health practitioner to listen to, and switch between the bodily sounds from multiple patients when multiple stethoscopes are connected to the application.

In a further embodiment, a method of performing auscultation using the audible stethoscope 100 of the present invention comprises initially placing a stethoscope bell/diaphragm of a stethoscope head against a person's body and positioned over a source of auscultation, such as the patient's heart, lungs or the like. An auscultation signal corresponding to the sound is then transmitted to the processing portion 108 for processing, amplification, filtering and then to the speaker 110 to be played aloud for the medical professional 502. A graphical image of the auscultation signal may also be shown on the display 307.

The audible stethoscope 100 of the present invention can be used for an adult, children, infant, etc., and may come in multiple colors. The material is such that patient comfort is assured while the audible stethoscope 100 is used for auscultation. The audible stethoscope 100 of the present invention is ergonomically designed such that it can be used by a single hand operation, and has a nonmetallic contact surface to be placed in contact with a patient's body.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “tubeless stethoscope”, “tubeless handheld stethoscope”, “tubeless auscultation device”, “stethoscope”, “audible handheld stethoscope” and “tubeless audible handheld stethoscope” are interchangeable and refer to the tubeless audible handheld stethoscope 100 of the present invention.

Notwithstanding the forgoing, the tubeless audible handheld stethoscope 100 of the present invention can be of any suitable size and configuration as is known in the art without affecting the overall concept of the invention, provided that it accomplishes the above stated objectives. One of ordinary skill in the art will appreciate that the size, configuration and material of the tubeless audible handheld stethoscope 100 as shown in the FIGS. are for illustrative purposes only, and that many other sizes and shapes of the tubeless audible handheld stethoscope 100 are well within the scope of the present disclosure. Although the dimensions of the tubeless audible handheld stethoscope 100 are important design parameters for user convenience, the tubeless audible handheld stethoscope 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical device comprising:
a receiving portion to collect a vibration or emission from a patient, wherein the receiving portion is comprised of a bell; and
a processing portion in communication with the receiving portion and comprised of an integrated speaker and a filter for filtering the vibration or emission received from the patient, wherein the receiving portion and the processing portion provide remote monitoring of the patient.

2. The medical device of claim 1, wherein the receiving portion is comprised to a microphone to collect the vibration or emission.

3. The medical device of claim 1, wherein the processing portion is comprised of an amplifier for amplifying the vibration or emission received from the patient.

4. The medical device of claim 1, wherein the processing portion is comprised of a microprocessor and a battery.

5. The medical device of claim 4, wherein the battery is a rechargeable battery.

6. The medical device of claim 1, wherein the processing portion is comprised of a memory module for recording the vibration or emission.

7. The medical device of claim 1, wherein the processing portion is comprised of a wireless transmission module.

8. The medical device of claim 1 further comprising a carrying clip.

9. The medical device of claim 1, wherein the medical device is sized and configured to fit within a pocket or a garment.

10. The medical device of claim 1 further comprising an LED indicator.

11. A handheld stethoscope comprising:
a bell comprised of a diaphragm concealing a microphone, wherein the bell is adapted to receive a vibration or emission from a patient;
a processing unit connected to the bell by a connecting assembly, wherein the processing unit is comprised of an amplifier and an integrated speaker; and
a microprocessor for converting the vibrations or emission into an audible sound for output on the integrated speaker.

12. The handheld stethoscope as recited in claim 11, wherein the processing unit further comprises a filter connected to the microprocessor for filtering the vibration or emission.

13. The handheld stethoscope as recited in claim 11 further comprising a battery for supplying power to the processing unit.

14. The handheld stethoscope as recited in claim 11, wherein the connecting assembly is comprised of a connecting tube and one or more connectors.

15. The handheld stethoscope as recited in claim 11, wherein the processing unit further comprises a memory and a wireless transmitter.

16. The handheld stethoscope as recited in claim 11 further comprising a power monitor to power down the handheld stethoscope after a period of non-use.

17. A portable stethoscope comprising:
a stethoscope having a bell for collecting a vibration signal from an area over an internal organ;
the bell comprising a microphone and being connected to a processing portion via a connecting tube, wherein the connecting tube transferring the vibration signal to an amplifier;
the amplifier is connected to a microprocessor, a filter and an internal speaker, and
the microprocessor and the filter control process the vibration signal to provide a sound to be output on the internal speaker.

18. The portable stethoscope as recited in claim 17, wherein the vibration signal is amplified to a desired frequency.

19. The portable stethoscope as recited in claim 17, wherein the microprocessor digitizes the vibration signal for transmission to a remote device by a transmitter in the processing portion.

20. The portable stethoscope as recited in claim 17 further comprising a LED display for displaying a signal corresponding to the vibration signal or a status of the portable stethoscope.
